(19) United States
(12) Patent Application Publication
(10) Pub. No.: US 2004/0110222 A1
(43) Pub. Date: Jun. 10, 2004

(54) Biopolymer detecting method and biochip

(75) Inventors: Kazuhisa Fukushima, Tokyo (JP); Nobuo Shimamoto, Mishima City (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: Yokogawa Electric Corporation, Tokyo (JP)

(21) Appl. No.: 10/727,510

(22) Filed: Dec. 5, 2003

(30) Foreign Application Priority Data
Dec. 5, 2002 (JP) 2002-353559

(51) Int. Cl. C12Q 1/68

(52) U.S. Cl. 435/6

(57) ABSTRACT

The present invention relates to biopolymer detection utilizing antigen-antibody reaction, intended to improve the S/N ratio, to increase the detection sensitivity, and to shorten the detection time.

According to the present invention, target biopolymers labeled with a fluorescent material and beads, onto the surface of which probe biopolymers and beads-ID recognizing address linkers are fixed, are put in a solution to hybridize the target biopolymers and the probe biopolymers, then the above address linkers are captured by antigen-antibody reaction using the addressing probe protein which is in such relation to the said address linkers as either one of the addressing probe protein and the address linkers is an antigen and the other is the corresponding antibody.

### BACKGROUND OF THE INVENTION

1. Field of the Invention

The present invention relates to a method of detecting biopolymers such as deoxyribonucleic acid (hereafter called DNA), ribonucleic acid (hereafter called RNA)(RNA is a transcription product from DNA, including messenger RNA (mRNA), ribosomal RNA (rRNA), transfer RNA (tRNA) or low molecular-weight RNA), protein, etc. and to biochips used for that method.

2. Description of the Prior Art

Techniques for decoding biopolymer structures (hereafter DNA is used as an example) using a micro array chip have been well known, for example, as mentioned in the gazette of Japanese Laid-open Patent Application No. 2000-131237. A micro array chip of this type for DNA is usually formed as described below to make it possible to decode the DNA structure.

Probe DNAs having a sequence complementary to the target mRNA (complementary DNA, hereafter called cDNA) are fixed by being spotted in an array on a glass (or plastic) substrate. The target mRNA (cDNA.) labeled with a fluorescent material is dropped onto the substrate. The probe and target having a sequence complementary to each other are bonded due to hybridization but those not having the sequence complementary to each other are not bonded.

After the hybridization has progressed sufficiently, the surface of the substrate is washed with washing buffer liquid to wash away the target which has not been hybridized. Next, as mentioned, for example, in the gazette of Japanese Laid-open Patent Application No. 2000-235035, the presence or absence of target mRNA (cDNA) and its quantity can be measured by optically reading the position of fluorescent labels and the amount of its fluorescence with a reader.

However, although conventional DNA micro arrays can provide objective data through an above-described series of protocols, there are actually various problems in the protocols in each step. As a result, there are many problems in the data obtained, such as accuracy, reproducibility, repeatability, sensitivity and others, and thus standardization of experimental data has not advanced and so DNA micro arrays have not become widely known in clinical sites along with problems in terms of contents.

The items specifically influential in various problems are S/N ratio, detection sensitivity, detection time, and reproducibility.

### SUMMARY OF THE INVENTION

The present invention intends to solve the above-described problems and its objective is to provide a biopolymer detecting method utilizing the antigen-antibody reaction aiming at improving the S/N ratio, increasing the detection sensitivity, and shortening the detection time, and to offer a biochip used for that method.

### BRIEF DESCRIPTION OF THE DRAWINGS

[FIG. 1]

FIG. 1 is a drawing illustrating the principle of the biopolymer detecting method of an embodiment of the present invention.

[FIG. 2]

FIG. 2 is another drawing illustrating the principle of the biopolymer detecting method of an embodiment of the present invention.

[FIG. 3]

FIG. 3 is another drawing illustrating the principle of the biopolymer detecting method of an embodiment of the present invention.

### DETAILED DESCRIPTION OF THE PREFERRED EMBODIMENTS

In the present invention, the advantages of beads and those of DNA arrays are combined. The advantages of beads are: many probe DNAs can be bonded because the surface areas per unit volume of beads are larger than those of flat plates, opportunities to encounter target biopolymers in a solution are increased tremendously because the beads can freely move in the solution, and thus a trace amount of target DNA in the solution can be captured with extremely high sensitivity (generally about 1000-fold or more of that of the DNA array).

On the other hand, however, beads have a disadvantage that each bead cannot be identified, that is, which DNA is bonded to which bead cannot be known. Although various trials are being carried out such that color beads are usually used or beads are identified using two-color light sources to recognize beads-ID, they include the problems that there are only few identifiable types and such equipment becomes complicated, expensive, and large, making it difficult to handle. The present invention cleverly overcomes these problems by enabling identification using the antigen-antibody reaction of proteins located on the beads and the array.

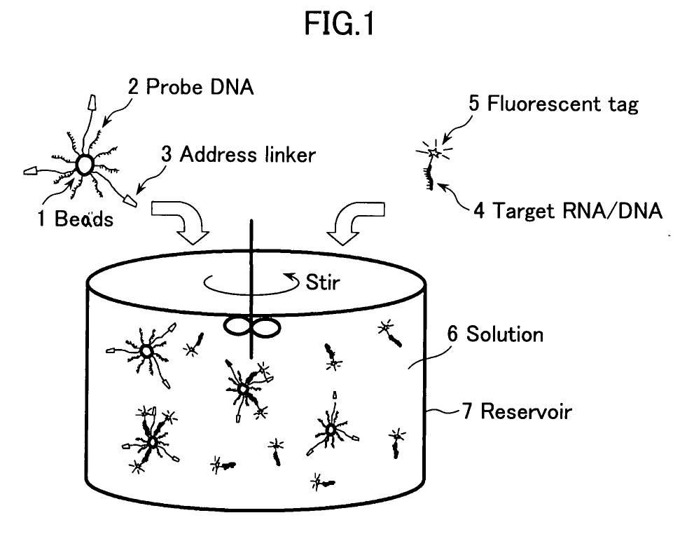

The present invention will be described in detail using drawings. FIGS. 1 to 3 are drawings illustrating the principle of the biopolymer detecting method of an embodiment of the present invention. This is hereby described for the case where the biopolymer is DNA.

As shown in FIG. 1, probe DNA 2 is fixed onto the surface of beads 1. As the beads, magnetic beads or beads made of metals or plastics can be employed.

In addition to the above, address linker 3 (address-judging antigen or address-judging antibody) for recognizing specific beads number ID is fixed on the surface of beads 1. On the other hand, RNA, cDNA or protein (hereafter these are represented by “RNA”) to be used as the target 4 is labeled with fluorescent tag 5.

The above-described beads 1, target RNA 4 and buffer solution 6 are put in reservoir 7 together and are stirred if necessary using a physical, electrical or chemical means. As a result, to probe DNA 2 located on the surfaces of beads 1, target RNA 4 is bonded, which is in complementary relation to probe DNA 2.

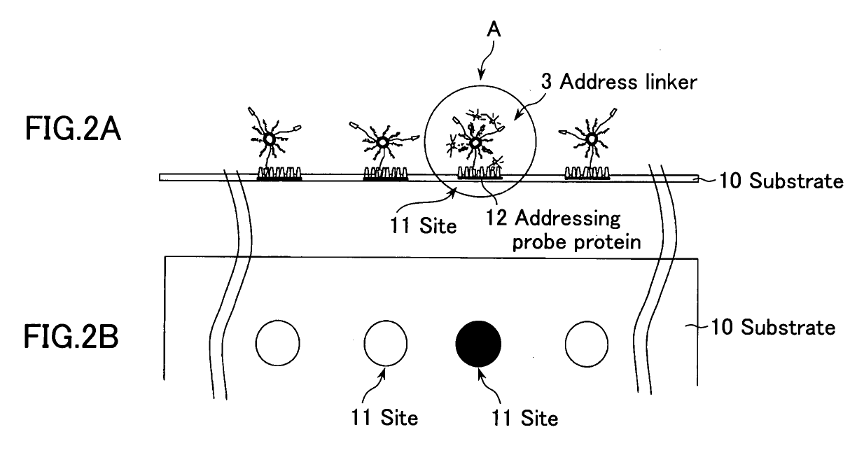

Next, the above beads on which target RNA 4 is bonded to probe DNA 2 are poured onto sites 11 arranged in an array of substrate 10. In FIGS. 2, drawing (a) indicates a side view and drawing (b) indicates a plan.

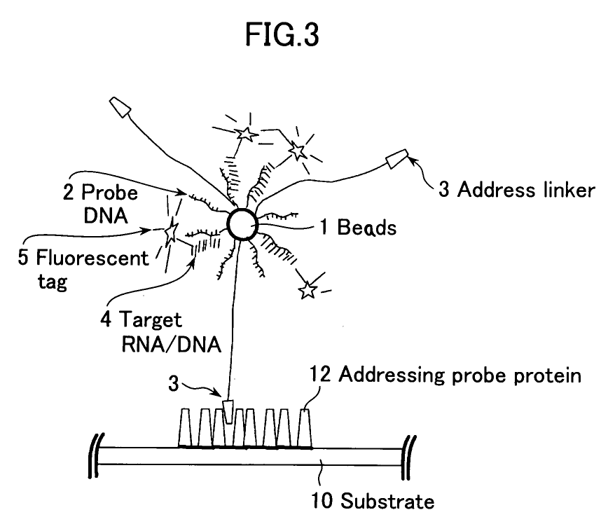

Addressing probe protein 12 for recognizing beads 1 ID by capturing ID-recognizing address linkers 3 located on the surfaces of beads 1 is fixed onto sites 11 in advance. Further, FIG. 3 is an enlarged drawing of part A enclosed with a circle in FIG. 2.

Address linker 3 is bonded to addressing probe protein 12 through antigen-antibody reaction. It is possible to recognize, by fluorescent tag 5, on which site 11 beads 1 are bonded to addressing probe protein 12. The fluorescent tag can be easily detected using a fluorescence reader (not shown in the drawing).

In such a manner as described above, the existence of target RNA 4 and its amount can be measured efficiently.

Furthermore, the above description merely shows a specific appropriate embodiment for the purpose of describing and indicating one example of the present invention. Accordingly, the present invention is not restricted to the above embodiment but may be embodied in many other specific forms, changes, and versions without departing from the spirit or essential characteristics thereof.

As described above, there are the following effects according to the present invention:

(1) Since beads have large surface areas, many probe DNAs can be bonded to beads. Accordingly, a trace amount of target biopolymers in a solution can be easily captured with an extremely high sensitivity (sensitivity of about 1000-fold or more the sensitivity obtained with general DNA arrays).

(2) Since target DNA can be hybridized and bonded to many probe DNAs bonded to one bead, the S/N ratio can be easily improved.

(3) Since the chance of target DNA encountering probe DNA increases through the fact that many probe DNAs are bonded to one bead and by stirring the solution, the detection time (mainly the time required for hybridization) can be easily shortened and, at the same time, the target DNA and the probe DNA can be hybridized with extremely high sensitivity.

What is claimed is:

1. A biopolymer detecting method in which target biopolymers are detected by capturing the target biopolymers on the substrate side, and that is characterized by a process in which target biopolymers labeled with a fluorescent material and beads, onto the surface of which probe biopolymers and beads-ID recognizing address linkers are fixed, are put in a solution to hybridize the target biopolymers and the probe biopolymers, then said address linkers are captured by antigen-antibody reaction using the addressing probe protein which is in such relation to said address linkers as either one is an antigen and the other is the corresponding antibody.

2. A biopolymer detecting method in accordance with claim 1, wherein said address linkers consist of a type of antigen or a type of antibody for address judgment to recognize said beads-ID.

3. A biopolymer detecting method in accordance with claim 1 or claim 2, wherein said target biopolymers and said beads are put in a reservoir together with a buffer solution and are stirred using a physical, electrical or chemical means.

4. A biopolymer detecting method in accordance with any of claims 1 to 3, wherein magnetic beads or beads made of metal or plastics are employed as said beads.

5. A biopolymer detecting method in accordance with any of claims 1 to 4, wherein said target biopolymers are RNAs which are transcription products from DNAs, or cDNAs, or proteins.

6. A biochip composed of addressing probe protein fixed onto a substrate, the protein being capable of capturing address linkers for ID recognition fixed onto the surface of beads using antigen-antibody reaction, together with probe biopolymers to be bonded to target biopolymers using the hybridization method.
